(19) United States
(12) Patent Application Publication
(10) Pub. No.: US 2022/0249354 A1
(43) Pub. Date: Aug. 11, 2022

(54) WATER BASED COMPOSITION

(71) Applicant: Shiseido Company, Ltd., Chuo-ku, Tokyo (JP)

(72) Inventor: Keita NISHIDA, Tokyo (JP)

(73) Assignee: Shiseido Company, Ltd., Chuo-ku, Tokyo (JP)

(21) Appl. No.: 17/629,639

(22) PCT Filed: Jul. 17, 2020
(86) PCT No.: PCT/JP2020/027855

§ 371 Date: Jan. 24, 2022

(30) Foreign Application Priority Data
Jul. 26, 2019 (JP) 2019-137492

(51) Int. Cl. A61K 8/891; A61K 8/06; A61K 8/81; A61K 8/19; A61K 8/37; A61K 8/40; A61K 8/86; A61Q 17/04; A61Q 1/12; A61Q 1/02

(52) U.S. Cl. CPC A61K 8/891; A61K 8/062; A61K 8/8147; A61K 8/19; A61K 8/37; A61K 2800/48; A61K 8/86; A61Q 17/04; A61Q 1/12; A61Q 1/02; A61K 8/40

(57) ABSTRACT

To provide a water based composition having an appropriate level of viscosity, which does not impart a sensation of stickiness, corrects unevenness such as pores, and has long lasting cosmetic effects. A water based composition includes an (A) crosslinked siloxane elastomer, a (B) lipophilic porous powder, and a (C) aqueous medium.

### CROSS-REFERENCE TO RELATED APPLICATIONS

This application is a National Phase Entry of PCT International Application No. PCT/JP2020/027855 filed on Jul. 17, 2020, which claims priority under 35 U.S.C § 119(a) to Japanese Patent Application No. 2019-137492 filed on Jul. 26, 2019.

### TECHNICAL FIELD

The present disclosure is related to a water based composition.

### BACKGROUND ART

Conventionally, there has been demand for a cosmetic having a high unevenness correcting effect that causes unevenness of skin, such as pores and fine wrinkles, to become less noticeable, thereby causing the skin to appear smooth. For example, PCT Japanese Phase Publication No. 2007-510691 discloses a cosmetic composition in which a crosslinked poly siloxane elastomer and light scattering particles are combined. This cosmetic composition forms a coating film that covers pores with the crosslinked poly siloxane elastomer, to cause the appearance of the pores to become flat.

In addition, Japanese Unexamined Patent Publication No. 2005-41795 discloses an oil based cosmetic having a superior unevenness correcting effect, by which wrinkles, pores, etc. are caused to be less noticeable, a natural appearing finish, a sensation of bare skin, and transparency. This oil based cosmetic employs a silicone powder constituted by a crosslinked silicone-web shaped silicone block copolymer and crosslinked polymethyl methacrylate (PMMA) powder together in a silicone gel base material. Further, PCT Japanese Phase Publication No. 2019-510783 discloses that a cosmetic composition that includes an oil wax gel that contains silicone oil and wax causes diffuse reflection of light at locations where wrinkles are at without the use of a powder, effectively concealing wrinkles of the skin, thereby temporarily hiding the wrinkles.

Meanwhile, compositions in which lipophilic porous powders are blended are known as compositions which are capable of correcting unevenness such as pores without employing inorganic powders that exhibit high concealing abilities, while having the appearance and usability of a skin care cosmetic composition (International Patent Publication No. WO 2017/073758 through 6, for example). Cosmetics that contain lipophilic porous powder absorb sebum which is secreted on the skin over time, which suppresses the occurrence of a sensation of stickiness and maintains a silky feel. In addition, spherical lipophilic porous powders also exhibit the effect of causing unevenness on the skin to become less visually noticeable.

Incidentally, if the compositions which are disclosed in International Patent Publication No. WO 2017/073758, International Patent Publication No. WO 2018/198737 and Japanese Unexamined Patent Publication No. 2017-165675 are to have a sensation of use similar to that of a beauty essence or a milky lotion, which are skin care cosmetics, it is preferable for the viscosities thereof to be low. Meanwhile, in the case of makeup cosmetics, there is a demand for higher viscosity. Generally, a thickener is added or increased in order to increase viscosity, but this tends to cause stickiness. If there is a sensation of stickiness after application, it is difficult to apply it evenly to the skin, the effect of correcting unevenness such as pores is reduced, and the effects of the cosmetic will not last long.

As a result of focused study by the present inventors, it was discovered that it is possible to synergistically improve viscosity while reducing stickiness by combining specific components, to arrive at the present disclosure.

That is, the present disclosure provides a water based composition having an appropriate level of viscosity, which does not impart a sensation of stickiness, corrects unevenness such as pores, and has long lasting cosmetic effects.

A water based composition of the present disclosure contains:

an (A) crosslinked siloxane elastomer;

a (B) lipophilic porous powder; and

a (C) aqueous medium.

It is preferable for a crosslinked polymethyl methacrylate to be the principal material of the (B) lipophilic porous powder.

It is preferable for the water based composition of the present disclosure to further contain a (D) water soluble thickener.

It is preferable for the water based composition of the present disclosure to further contain a (E) surfactant, and for the surfactant to be a poly oxyethylene-poly oxy alkylene alkyl ether block polymer having an HLB value within a range from 9 to 17 or a poly oxyethylene fatty acid glyceryl.

The water based composition of the present disclosure may further contain a (F) polar oil component.

The (F) polar oil component may be a UV ray absorber.

It is preferable for the ratio of the (B) lipophilic porous powder with respect to the (C) aqueous medium to be greater than or equal to 0.1.

The water based composition of the present disclosure may be a oil in water type composition.

### DETAILED DESCRIPTION OF THE PREFERRED EMBODIMENTS

The water based composition of the present disclosure contains:

an (A) crosslinked siloxane elastomer;

a (B) lipophilic porous powder; and

a (C) aqueous medium.

Therefore, the water based composition is that which corrects unevenness such as pores while not imparting a sensation of stickiness even with an appropriate level of viscosity, and has long lasting cosmetic effects.

### EMBODIMENTS OF THE DISCLOSURE

The water based composition of the present disclosure will be described in detail below. The water based composition of the present disclosure contains:

an (A) crosslinked siloxane elastomer;

a (B) lipophilic porous powder; and

a (C) aqueous medium.

Each of the above components will be described in detail. Note that with respect to the components, there will be cases in which they are referred to simply as “component (A)”, “component (B)”, etc. In addition, in the present specification, PEG is an abbreviation for polyethylene glycol, PPG is an abbreviation for polypropylene glycol, EO is an abbreviation for ethylene oxide, PO is an abbreviation for propylene oxide, POE is an abbreviation for poly oxyethylene, POP is an abbreviation for poly oxy propylene, and VP is an abbreviation for vinyl pyrrolidone.

### (A) Crosslinked Siloxane Elastomer

The (A) crosslinked siloxane elastomer which is blended in the water based composition of the present disclosure is a siloxane elastomer (silicone elastomer) in which poly dimethyl siloxane is three dimensionally crosslinked, and includes those which are emulsified and those which are not emulsified.

The emulsified crosslinked siloxane elastomer is not particularly limited, and examples thereof include: poly oxyethylene methyl poly siloxane. alkyl group containing crosslinked poly oxyethylene methyl poly siloxane, crosslinked poly glycerin denatured silicone, alkyl group containing crosslinked poly glycerin denatured silicone, etc. Elastomers which are commercially available as swelling agents in various oils such as silicone oil, mineral oil, triethyl hexanoin, squalene, etc. may be employed as the emulsified crosslinked siloxane elastomer. Specific examples thereof are listed below.

Examples of poly oxyethylene methyl poly siloxane cross polymer swelling agents include KSG-210 (((PEG-10/15)/dimethicone) cross polymer; a dimethicone mixture in which 20 to 30% of the contents are crosslinked) (by Shin Etsu Chemical Industries, K. K.), 9011 silicone elastomer blend ((PEG-12/dimethicone) cross polymer; a cyclomethicone mixture) (by Toray Dow Corning K. K.), etc.

Examples of alkyl group containing poly oxyethylene methyl poly siloxane cross polymer swelling agents include KSG-310 ((PEG-15/lauryl dimethicone) cross polymer; a mineral oil mixture in which 25 to 35% of the contents are crosslinked), KSG-320 ((PEG-15/lauryl dimethicone) cross polymer; a isododecane mixture in which 20 to 30% of the contents are crosslinked), KSG-330 ((PEG-15/lauryl dimethicone) cross polymer; a triethyl hexanoin mixture in which 15 to 25% of the contents are crosslinked), KSG-340 ((PEG-15/lauryl dimethicone) cross polymer and (PEG-10/lauryl dimethicone cross polymer; a squalene mixture in which 25 to 30% of the contents are crosslinked) (all by Shin Etsu Chemical Industries, K. K.), etc.

Examples of poly glycerin denatured silicone cross polymer swelling agents include KSG-710 ((dimethicone/poly glycerin-3) cross polymer; a dimethicone mixture in which 20 to 30% of the contents are crosslinked) (by Shin Etsu Chemical Industries, K. K.), etc.

Examples of alkyl group containing poly glycerin denatured silicone cross polymer swelling agents include KSG-810 ((lauryl dimethicone/poly glycerin 3) cross polymer; a mineral oil mixture in which 25 to 35% of the contents are crosslinked), KSG-820 ((lauryl dimethicone/poly glycerin 3) cross polymer; an isododecane mixture in which 20 to 30% of the contents are crosslinked), KSG-830 ((lauryl dimethicone/poly glycerin 3) cross polymer; a triethyl hexanoin mixture in which 15 to 25% of the contents are crosslinked), KSG-840 ((lauryl dimethicone/poly glycerin 3) cross polymer; a squalene mixture in which 25 to 35% of the contents are crosslinked) (all by Shin Etsu Chemical Industries, K. K.).

The non emulsified crosslinked siloxane elastomer is not particularly limited, and examples thereof include methyl poly siloxane cross polymers, methyl phenyl poly siloxane cross polymers, vinyl dimethicone/lauryl dimethicone cross polymers, lauryl poly dimethyl siloxy ethyl dimethicone/bis-vinyl dimethicone cross polymers, alkyl (C30-C40) stearyl dimethicone cross polymers, stearyl dimethicone cross polymers, etc. Elastomers which are commercially available as swelling agents in various oils such as silicone oil, mineral oil, triethyl hexanoin, squalene, etc. may be employed as the non emulsified crosslinked siloxane elastomer. Specific examples thereof are listed below.

Examples of methyl poly siloxane cross polymer swelling agents include dimethicone cross polymer swelling agents such as 9040 silicone elastomer blend (a mixture of dimethicone cross polymer and cyclopenta siloxane, in which 12 percent of the contents are crosslinked), 9041 silicone elastomer blend (a mixture of dimethicone cross polymer and dimethicone 5 mPa·s, in which 16 percent of the contents are crosslinked), 9045 silicone elastomer blend (a mixture of dimethicone cross polymer and cyclopenta siloxane, in which 12.5 percent of the contents are crosslinked), and EL-8040ID silicone organic blend (a mixture of dimethicone cross polymer and isododecane, in which 18% of the contents are crosslinked) (all by Toray Dow Corning K. K.), and dimethicone/vinyl dimethicone cross polymer swelling agents, such as KSG-15 ((dimethicone/vinyl dimethicone) cross polymer; a cyclopenta siloxane mixture in which 4% to 10% of the contents are crosslinked), KSG-16 ((dimethicone/vinyl dimethicone) cross polymer; a dimethicone 6 mPa·s mixture in which 20% to 30% of the contents are crosslinked), KSG-1610 ((dimethicone/vinyl dimethicone) cross polymer; a methyl trimethicone mixture in which 15% to 20% of the contents are crosslinked) (all by Shin Etsu Chemical Industries, K. K.).

KSG-18A ((dimethicone/phenyl vinyl dimethicone) cross polymer; a diphenyl siloxy phenyl trimethicone mixture in which 10% to 20% of the contents are crosslinked) (by Shin Etsu Chemical Industries, K. K.) is an example of a methyl phenyl poly siloxane cross polymer swelling agent.

Examples of vinyl dimethicone/lauryl dimethicone cross polymer swelling agents include KSG-41A ((vinyl dimethicone/lauryl dimethicone) cross polymer; a mineral oil mixture in which 20% to 30% of the contents are crosslinked), KSG-42A ((vinyl dimethicone/lauryl dimethicone) cross polymer; an isododecane mixture in which 15% to 25% of the contents are crosslinked), KSG-43 ((vinyl dimethicone/lauryl dimethicone) cross polymer; a triethyl hexanoin mixture in which 25% to 35% of the contents are crosslinked), and KSG-44 ((vinyl dimethicone/lauryl dimethicone) cross polymer; a squalene mixture in which 25% to 35% of the contents are crosslinked) (all by Shin Etsu Chemical Industries, K. K.).

Examples of lauryl poly dimethyl siloxane dimethicone/bis-vinyl dimethicone cross polymer swelling agents include KSG-042Z ((lauryl poly dimethyl siloxy ethyl dimethicone/bis-vinyl dimethicone) cross polymer; an isododecane mixture in which approximately 20% of the contents are crosslinked) and KSG-045Z ((lauryl poly dimethyl siloxane dimethicone/bis-vinyl dimethicone) cross polymer; a cyclo penta siloxane mixture in which approximately 20% of the contents are crosslinked) (both by Shin Etsu Chemical Industries, K. K.).

Examples of alkyl (C30 to C45) stearyl dimethicone cross polymer swelling agents include VELVESIL 125 (alkyl (C30 to C45) stearyl dimethicone cross polymer; a cyclo penta siloxane mixture in which approximately 12.5% of the contents are cross linked) and VELVESIL 034 (alkyl (C30 to C45) stearyl dimethicone cross polymer; a capryl methicone mixture in which approximately 16% of the contents are cross linked) (both by Momentive Performance Materials Co.).

Examples of stearyl dimethicone cross polymer swelling agents include VELVESIL DM stearyl dimethicone cross polymer; a dimethicone 5 mPa·s mixture in which approximately 17% of the contents are cross linked) (by Momentive Performance Materials Co.).

It is preferable for the amount of the (A) component to be blended to be within a range from 0.01 to 20 percent by mass, more preferably a range from 0.01 to 10 percent by mass, further preferably a range from 0.01 to 8 percent by mass, and most preferably a range from 0.5 to 8 percent by mass with respect to the total amount of the water based composition. By the amount of the (A) component being greater than or equal to 0.01 percent by mass, viscosity can be more synergistically be improved by being combined with the (B) component to be described below. In addition, by the amount of the (A) component being less than or equal to 20 percent by mass, the sensation of stickiness can be reduced to a greater degree.

The (A) component may be one or a combination of two or more elastomers which are selected from among the emulsified crosslinked siloxane elastomer and the non emulsified crosslinked siloxane elastomer which are described above.

### (B) Lipophilic Porous Powder

Examples of the lipophilic porous powder to be blended in the water based composition of the present disclosure include, for example, crosslinked polymethyl methacrylate, porous nylon powder, etc. These powders may contain the other components to be described below, for example, silica, at an amount of less than or equal to several percent by mass. It is preferable for the lipophilic porous powder to have crosslinked polymethyl methacrylate as its principal component. The expression “principal component” means that the lipophilic porous powder may contain other components at an amount of less than or equal to several percent by mass, as described above. The average particle size is not particularly limited in the case that the lipophilic porous powder is spherical. However, it is preferable for the average particle size to be within a range from 0.1 to 100 μm, more preferably a range from a range from 0.5 to 50 μm, and still more preferably a range from 1 to 20 μm.

A material which is lipophilic, or a material which is hydrophilic but is coated by a lipophilic substance, silica or the like which has undergone a process to become hydrophobic, for example, may be employed. In the case that a coated material is employed, it is preferable for the interiors of pores to be processed to become lipophilic as well.

It is preferable for the maximum amount of oil which is capable of being absorbed by the lipophilic porous powder to be greater than or equal to 100 percent by mass with respect to the powder. This is because the maximum amount of oil which is capable of being absorbed is closely related to the pore volume, and the porous aspect influences the light diffusing properties of the lipophilic porous powder. By the maximum amount of oil which is capable of being absorbed by the lipophilic porous powder being greater than or equal to 100 percent by mass with respect to the powder, a sufficient light diffusing effect can be exhibited and a sufficient pore correcting effect can be achieved.

It is preferable for the amount of the lipophilic porous powder to be blended to be within a range from 0.5 to 30 percent by mass, and more preferably a range from 0.5 to 20 percent by mass with respect to the total amount of the water based composition. By the amount of the lipophilic porous powder being greater than or equal to 0.5 percent by mass, viscosity can be improved more appropriately, while a greater pore correcting effect and an effect of a longer lasting sensation of smoothness can be exhibited. By the amount of the lipophilic porous powder being less than or equal to 30 percent by mass, favorable powder dispersion properties can be secured.

### (C) Aqueous Medium

The aqueous medium of the present disclosure may be a medium that contains water as a principal component. The aqueous medium may be water alone, or may contain a lower alcohol and/or a polyhydric alcohol. That water is the principal component of the aqueous medium means that the amount of water in the aqueous medium is greater than or equal to 50 percent by mass of the aqueous medium.

The water in the present disclosure may be ion exchanged water, purified water, or tap water.

Examples of lower alcohols include, for example, ethanol, propanol, isopropanol, etc.

Examples of polyhydric alcohols include, for example, dihydric alcohols such as ethylene glycol, propylene glycol, trimethylene glycol, 1,2-butylene glycol, 1,3-butylene glycol, tetramethylene glycol, and 2,3-butylene glycol; and trihydric alcohols such as glycerin.

It is preferable for the percentage of the lower alcohol and the polyhydric alcohol within the aqueous medium to be less than or equal to 30 percent by mass, and more preferable less than or equal to 25 percent by mass.

It is preferable for the amount of the (C) aqueous medium to be blended in the water based composition of the present disclosure to be within a range from 10 to 80 percent by mass with respect to the total amount of the water based composition. By the amount of the (C) aqueous medium being greater than or equal to 10 percent by mass, the dispersion properties of the (B) component in the water based composition will become more favorable, and by the amount of the (C) aqueous medium being less than or equal to 80 percent by mass, a cosmetic lasting effect can be improved.

It is preferable for the ratio of the (B) lipophilic porous powder with respect to the (C) aqueous medium to be greater than or equal to 0.01, more preferably a range from 0.01 to 2, and still more preferably a range from 0.1 to 1. By the ratio of the (B) lipophilic porous powder with respect to the (C) aqueous medium being greater than or equal to 0.01, the viscosity of the water based composition can be further improved.

### (D) Water Soluble Thickener

The viscosity of the water based composition of the present disclosure can be improved by the (A) component and the (B) component. The water based composition may further contain a (D) water soluble thickener in order to improve dispersion properties. Water soluble thickeners which are generally utilized in cosmetics may be employed as the (D) water soluble thickener.

Examples of the water soluble thickener include, for example, plant based polymers such as gum Arabic, tragacanth, galactan, carob gum, guar gum, karaya gum, carrageenan, xanthan gum, starch, agar, quince seed (Cydonia oblonga), algae colloid (brown algae extract), microbial polymers such as dextran, succinoglucan, and purulan, animal based polymers such as collagen, casein, albumin, gelatin, and starch based polymers such as starch (rice, corn, potato, and wheat), carboxymethyl starch, methyl hydroxy propyl starch, etc.

Other examples of the water soluble thickener include cellulose based polymers such as methyl cellulose, nitro cellulose, ethyl cellulose, methyl hydroxypropyl cellulose, hydroxyethyl cellulose, sodium cellulose sulfate, hydroxypropyl cellulose, sodium carboxymethyl cellulose, crystalline cellulose, and cellulose powder, and alginic acids such as sodium alginate, propylene glycol ester alginate cellulose, etc.

Further examples of the water soluble thickener include vinyl based polymers such as polyvinyl methyl ether and carboxy vinyl polymer, poly oxyethylene based polymers, poly oxyethylene poly oxy propylene copolymer based polymers, acrylic polymers such as poly ethyl acrylate and polyacrylamide, inorganic aqueous polymers such as polyethylene imine, cationic polymers, bentonite, magnesium aluminum silicate, laponite, hectorite, and silicic anhydride, PEG-240/decyltetradeceth-20/hexamethylene diisocyanate copolymer, (dimethyl lacrylamide/acryloyl dimethyl taurine Na)) cross polymer, (Na acrylate/acryloyl dimethyl taurine) copolymer, (alkyl acrylate/steares methacrylate-20) copolymer, (acryloyl dimethyl taurine ammonium/VP) copolymer, etc.

It is preferable for the amount of the (D) water soluble thickener to be blended in the water based composition of the present disclosure to be within a range from 0.005 to 2 percent by mass with respect to the total amount of the water based composition. By the amount of the (D) component being greater than or equal to 0.005 percent by mass with respect to the total amount of the water based composition, dispersion properties and dispersion stability can be improved. In addition, by the amount of the (D) component being less than or equal to 2 percent by mass with respect to the total amount of the water based composition, a sensation of stickiness can be suppressed.

### (E) Surfactant

In the case that a surfactant is blended in the water based composition of the present disclosure (in the case that the water based composition is to be of the oil in water type, for example), it is preferable for a poly oxyethylene-poly oxy alkylene alkyl ether block polymer or a poly oxyethylene fatty acid glyceryl having an HLB value within a range from 9 to 17 to be employed, from the viewpoint of having a small amount of adsorption to the (B) component while contributing greatly to emulsification, in other words, securing emulsion stability with a small amount of the surfactant.

The HLB value is calculated by Formula 1 below (wherein MW represents the molecular weight of hydrophilic groups and MO represents the molecular weight of lipophilic groups). An additive property is recognized for the HLB value, and the HLB value as a whole in the case that two or more types of nonionic surfactants are utilized in combination is represented by a weighted average of the HLB values of the individual surfactants.

HLB=7+11.7·log(MW/MO) [Formula 1]

(Poly Oxyethylene-Poly Oxy Alkylene Alkyl Ether Block Polymer Having an HLB Value within a Range from 9 to 17)

The poly oxyethylene-poly oxy alkylene alkyl ether block polymer represented by General Formula (1) below is an example of the poly oxyethylene-poly oxy alkylene alkyl ether block polymer.

R₁O—(PO)ₘ-(EO)ₙ—H (1)

In General Formula 1, R₁ is a hydrocarbon group having a carbon number within a range from 16 to 18, and is preferably a saturated or an unsaturated aliphatic hydrocarbon group. Examples of such aliphatic hydrocarbon groups include palmityl, stearyl, isostearyl, oleyl, linoril, etc.

PO is an oxy propylene group, and EO is an oxyethylene group.

In General Formula 1, PO and EO must be bonded in the state of blocks. Compositional stability cannot be sufficiently obtained in the case that PO and EO are bonded randomly. Note that the order in which propylene oxide and ethylene oxide are added is not particularly limited. In addition, the block state includes not only doubly stacked blocks, but triply and greater stacked blocks.

In General Formula (1), m and n respectively represent an added number of mols of PO and EO. 70>m>4, 70>n>10, and n>m.

It is preferable for the molecular weight of the block polymer of General Formula (1) to be greater than or equal to 800, and more preferably greater than or equal to 1500. The effects exhibited by the surfactant will be insufficient if the molecular weight is less than 800. In addition, although the upper limit of the molecular weight is not specified, there is a tendency for a sensation of stickiness to occur as the molecular weight becomes greater.

Examples of the block polymer represented by General Formula (1) include “Nikkol PBC44” (HLB value: 12.5), “Nikkol PBC33” (HLB value: 10.5), and “Nikkol PBC34” (HLB value: 16.5), all by Nikko Chemicals K. K.

(Poly Oxyethylene Aliphatic Glyceryl having an HLB value within a range from 9 to 17) Examples of the poly oxyethylene aliphatic glyceryl include, for example, POE glyceryl monoisostearate (8E.O.) (=PEG-8 glyceryl monoisostearate) [“EMALEX GWIS-108”, HLB value: 10, by Nippon Emulsion Co.], POE (20) glyceryl monoisostearate (=monoisostearate) PEG-20 glyceryl acid) [“EMALEX GWIS-120”, HLB value: 13, by Nippon Emulsion Co.], POE (7) coconut oil fatty acid glyceryl (=coconut oil fatty acid PEG-7 glyceryl) [“Cetiol HE”, HLB value: 13, by Cognis Japan Co.], etc.

It is preferable for the amount of the (E) component to be blended in the water based composition to be within a range from 0.01 to 5 percent by mass, and more preferably a range from 0.5 to 3 percent by mass with respect to the total amount of the water based composition. By the amount of the (E) component being greater than or equal than 0.01 percent by mass and less than or equal to 5 percent by mass, a greater emulsion stability can be secured.

### (F) Polar Oil Component

The water based composition of the present disclosure may contain an (F) polar oil component. By the surfactant of the (E) component being blended, emulsion stability can be improved further, even in the case that the polar oil component is blended in the water based composition.

Although the IOB value of the polar oil component is not particularly limited, it is preferable for the IOB value to be within a range from 0.05 to 0.80.

Note that the IOB value is an abbreviation for “Inorganic/Organic Balance” that represents the ratio of an inorganic value with respect to an organic value, and is an index that represents the degree of polarity of an organic compound. Specifically, the IOB value is represented as:

IOB value=Inorganic Value/Organic Value

Here, with respect to the “inorganic value” and the “organic value”, an “inorganic value” and an “organic value” are set according to each type of atom or functional group, such as an “organic value” of 20 for each carbon atom within a molecule and an “inorganic value” of 100 for each hydrocarbon group within a molecule, for example. The IOB value can be calculated by integrating the “inorganic value” and the “organic value” of all of the atoms and functional groups within an organic compound (refer to Fujita, Chemical Regions, Vol. 11, No. 10, pp. 719-725, 1957, for example).

Representative examples of the polar oil component include ester oils and UV ray absorbers.

Examples of ester oils include tripropylene glycol dineopentanate, isononyl isononanoate, isopropyl myristate, cetyl octanoate, octyl dodecyl myristate, isopropyl palmitate, butyl stearate, hexyl laurate, myristyl myristate, decyl oleate, hexyl decyl dimethyl octanoate, cetyl lactate, myristyl lactate, lanolin acetate, isocetyl stearate, isocetyl isostearate, cholesteryl 12-hydroxy stearate, cetyl ethyl hexanate, ethylene glycol di-2-ethyl hexanoate, dipenta erythritol fatty acid ester, N-alkyl glycol monoisostearate, neopentyl glycol dicaprate, diisostearyl malate, glycerin di-2-heptylundecanoate, trimethylolpropane tri-2-ethyl hexanoate, trimethylolpropane triisostearate, penta erythrityl tetra-2-ethyl hexanoate, triethylhexanoin (glycerin tri-2-ethyl hexanoate), glycerin trioctanoate, glycerin triisopalmitate, trimethylolpropane triisostearate, cetyl 2-ethyl hexanoate, 2-ethyl hexyl palmitate, glycerin trimyristate, glyceride tri 2-heptylundecanoate, castor oil fatty acid methyl ester, oleyl oleate, acetoglyceride, 2-heptylundecyl palmitate, diisobutyl adipate, N-lauroyl-L-glutamate-2-octyl dodecyl ester, di-2-heptylundecyl adipate, ethyl laurate, di-2-ethyl hexyl sebacate, 2-hexyl decyl myristate, 2-hexyl decyl palmitate, 2-hexyl decyl adipate, diisopropyl sebacate, 2-ethyl hexyl succinate, triethyl citrate, etc.

There are a great number of oil based UV ray absorbers having high polarity which are generally employed in cosmetics, and the UV ray absorber to be employed in the water based composition of the present disclosure is not particularly limited. Examples of such oil based UV ray absorbers include benzoic acid derivatives, salicylic acid derivatives, silicic acid derivatives, dibenzoylmethane derivatives, (3,3-diphenyl acrylate derivatives, benzophenone derivatives, benzylidene camphor derivatives, phenyl benzo imidazole derivatives, triazine derivatives, phenyl benzotriazole derivatives, anthranyl derivatives, imidazoline derivatives, benzal malonate derivatives, and 4,4-diarylbutadiene derivatives. Specific examples and product names are listed below. However, the UV ray absorber to be blended in the water based composition of the present disclosure is not limited to those listed below.

Examples of benzoic acid derivatives include para-amino benzoic acid ethyl, ethyl-dihydroxy propyl PABA, ethyl hexyl-dimethyl PABA (“Escalol 507” by ISP Chemicals, for example), glyceryl PABA, PEG-25-PABA (“Uvinal P25” by BASF Co., for example), diethyl amino hydroxy benzoyl benzoic acid hexyl (“Uvinal A Plus”, for example).

Examples of salicylic acid derivatives include homosalate (“Eusolex HMS” by Lona/EM Industries Co., for example), ethyl hexyl salicylate (“Neo Heliopan OS” by Harman & Reimer Co., for example), dipropylene glycol salicylate (“Dipsal” by Skel Co., for example), TEA salicylate (“Neo Heliopan TS” by Harman and Reimer Co., for example), etc.

Examples of silicic acid derivatives include octyl methoxycinnamate or ethyl hexyl methoxycinnamate (“Pulsol MCX” by Hoffmann-La Roche Co., for example), isopropyl methoxycinnamate, isoamyl methoxycinnamate (“Neo Heliopan E1000” by Herman and Reimer Co., for example), synnoxate, DEA methoxycinnamate, di isopropyl methyl silicate, glyceryl-ethyl hexanoate-di methoxycinnamate, di-(2-ethyl hexyl)-4′-methoxybenzalmalonate, etc.

4-tert-butyl-4′-methoxydibenzoylmethane (“Pulsol 1789”, for example) is an example of a dibenzoyl methane derivative.

Octocrylene (“Ubinal N539” by BASF, for example) is an example of a (3, (3-diphenyl acrylate derivative.

Benzophenone derivatives include benzophenone-1 (“Uvinal 400” by BASF Co.), benzophenone-2 (“Uvinal D50” by BASF Co., for example), benzophenone-3 or oxybenzone (“Uvinal M40” by BASF, for example), benzophenone-4 (“Uvinal MS40” by BASF Co., for example), benzophenone-5, benzophenone-6 (“Helisorb 11” by Norquay Co., for example), benzophenone-8 (“Spectra-Sorb UV-24” by American Cyanamide Co., for example), benzophenone-9 (“Uvinal DS-49” by BASF Co., for example), benzophenone-12, etc.

Examples of benzylidene camphor derivatives include 3-benzylidene camphor (“Mexoryl SD” by Chimex Co., for example), 4-methyl benzylidene camphor, benzylidene camphor sulfonate (“Mexoryl SL” by Chimex Co., for example), camphor benzalkonium methosulfate (“Mexoryl SO” by Chimex Co., for example), terephthaliledene di camphor sulfonate (“Mexoryl SX” by Chimex Co., for example), polyacrylamide methyl benzylidene camphor (“Mexoryl SW” by Chimex Co., for example), etc.

Examples of phenyl benzimidazole derivatives include phenyl benzimidazole sulfonate (“Eusolex 232” by Merck Co., for example), disodium phenyl dibenzo imidazole tetra sulfonate (“Neo Heliopan AP” by Herman & Reimer Co.), etc.

Examples of triazine derivatives include anisotriazine (“Tinosorb S” by Ciba Specialty Chemicals Co., for example), ethyl hexyl triazone (“Uvinal T150” by BASF Co., for example), diethyhexyl butamide triazone (“Uvasorb HEB” by Sigma 3V Co. for example), 2,4,6-tris (diisobutyl-4′-aminobenzalmaronate)-s-triazine, etc.

Examples of phenyl benzotriazole derivatives include drometrizole trisiloxane (“Silatrizole” by Rhodia Chimie Co., for example), methylene bis-(benzo triazolyl tetramethyl butylphenol) (“Tinosorb M” by Ciba Specialty Chemicals Co.), etc.

Menthyl anthranilate (“Neo Heliopan MA” by Harman & Reimer Co., for example) is an example of an anthranyl derivative.

Ethyl hexyl dimethoxy benzylidene dioxoimidazolin propionate is an example of an imidazoline derivative.

Poly organo siloxane having a benzal malonate functional group (polysilicone-15 “Pulsol SLX” by DSM Nutrition Japan Co., for example) is an example of a benzal malonate derivative.

1,1-dicarboxy (2,2′-dimethyl propyl)-4,4-diphenyl butadiene is an example of a 4,4-diaryl butadiene derivative.

The amount of the (F) polar oil component which is blended in the water based composition of the present disclosure may be adjusted as appropriate according to the properties thereof and intended use. In the case that the composition is to be an oil in water type composition, it is preferable for the amount of the (F) polar component to be within a range from 1 to 40 percent by mass, more preferably a range from 1 to 30 percent by mass, and still more preferably a range from 5 to 25 percent by mass with respect to the total amount of the water based composition. It is preferable for the amount of the UV ray absorber which is blended in the (F) polar oil component to be greater than or equal to 40 percent by mass, more preferably greater than or equal to 50 percent by mass, and still more preferably greater than or equal to 60 percent by mass of the polar oil component. The entirety of the polar oil component may be a UV ray absorber. By the amount of the (F) polar oil component being greater than or equal to 1 percent by mass, whitening due to the porous powder can be prevented, and by the amount of the (F) polar oil component being less than or equal to 40 percent by mass, a sensation of stickiness can be suppressed to a greater degree.

One or a combination of two or more components which are generally employed in cosmetic and external pharmaceutical preparations, such as moisturizers, powder components (other than the (A) component), liquid oils and fats, solid oils and fats, wax, hydrocarbon oils, polyhydric alcohols, surfactants (other than the (E) component), and saccharides may also be blended in the water based composition of the present disclosure. Components which may be blended in the water based composition of the present disclosure will be exemplified below.

Examples of moisturizers include, for example, polyethylene glycol, propylene glycol, glycerin, 1,3-butylene glycol, xylitol, sorbitol, maltitol, chondroitin sulfate, hyaluronic acid, mucoitin sulfate, caloninic acid, atelocollagen, sodium lactate, bile salt, d,l-pyrrolidone carboxylate salt, short chain soluble collagen, diglycerin (EO) PO adduct, isabiida extract, bupleurum extract, merylort extract, etc.

Examples of the powder component other than the (A) component include an inorganic powder (for example, talc, kaolin, mica, sericite, muscovite, phlogopite, synthetic mica, red mica, biotite, vermiculite, magnesium carbonate, calcium carbonate, aluminum silicate, barium silicate, calcium silicate, magnesium silicate, strontium silicate, tungstenate metal salt, magnesium, silica, zeolite, barium sulfate, calcined calcium sulfate (baked gypsum) calcium phosphate, fluorine apatite, hydroxy apatite, ceramic powder, metal soap (for example, zinc myristate, calcium palmitate, and aluminum stearate), boron nitride, etc.); an organic powder (for example, polyamide resin powder (nylon powder), polyethylene powder, methyl polymethacrylate powder, polystyrene powder, a styrene-acrylate copolymer resin powder, benzo quanamine resin powder, poly tetra fluorinated ethylene powder, cellulose powder, etc.); an inorganic white pigment (for example, titanium dioxide, zinc oxide, etc.); an inorganic red type pigment (for example, iron oxide, iron titanate, etc.); an inorganic purple type pigment (for example, manganese violet, cobalt violet, etc.); an inorganic green pigment (for example, chromium oxide, chromium hydroxide, cobalt titanate, etc.); an inorganic blue type pigment (for example, ultramarine, Prussian blue, etc.); a pearl pigment (for example, titanium oxide coated mica, titanium oxide coated bismuth oxychloride, titanium oxide coated talc, colored titanium oxide coated mica, bismuth oxychloride, fish scale foil, etc.); a metal powder pigment (for example, aluminum powder, copper powder, etc.); an organic pigment such as zirconium, barium, and aluminum flakes (for example, organic pigments such as red number 201, red number 202, red number 204, red number 205, red number 220, red number 226, red number 228, red number 405, orange number 203, orange number 204, yellow number 205, yellow number 401, and blue number 404, red number 3, red number 104, red number 106, red number 227, red number 230, red number 401, red number 505, orange number 205, yellow number 4, yellow number 5, yellow number 202, yellow number 203, green number 3, and blue number 1); natural color (for example, chlorophyll, β-carotene, etc.); etc.

However, in the case that an inorganic powder having a high refractive index (a refractive index of greater than or equal to 2, for example) is blended, it is preferable for the content of such an inorganic powder to be less than or equal to 5 percent by mass, and more preferably less than or equal to 1 percent by mass with respect to the total amount of the water based composition.

Examples of liquid oils and fats include, for example, avocado oil, camellia oil, turtle oil, macadamia nut oil, corn oil, mink oil, olive oil, rapeseed oil, egg yolk oil, sesame oil, persic oil, wheat germ oil, camellia sasanqua oil, castor oil, linseed oil, safflower oil, cottonseed oil, perilla seed oil, soybean oil, peanut oil, tea seed oil, kaya oil, rice bran oil, cinnamon oil, Japanese Paulonia oil, jojoba oil, germ oil, triglycerin, etc.

Examples of solid oils and fats include, for example, cacao butter, coconut oil, horse fat, hardened coconut oil, palm oil, beef fat, sheep fat, hardened beef fat, palm kernel oil, pork fat, beef bone fat, wolfberry kernel oil, hardened oil, beef leg fat, wolfberry, hardened castor oil, etc.

Examples of waxes include, for example, beeswax, candelilla wax, cotton wax, carnauba wax, bayberry wax, rosemary wax, spermaceti, montan wax, bran wax, lanolin, kapok wax, lanolin acetate, liquid lanolin, sugarcane wax, isopropyl lanolin fatty acid, hexyl laurate, reduced lanolin, Burrow, hard lanolin, shellac wax, POE lanolin alcohol ether, POE lanolin alcohol acetate, POE cholesterol ether, lanolin fatty acid polyethylene glycol, POE hydrogenated lanolin alcohol ether, etc.

Examples of hydrocarbon oils include, for example, liquid paraffin, ozokerite, pristane, paraffin, ceresin, squalene, petrolatum, microcrystalline wax, etc.

Examples of the higher fatty acids include, for example, lauric acid, myristic acid, palmitic acid, stearic acid, behenic acid, oleic acid, undecylenic acid, tallic acid, isostearic acid, linoleic acid, linolenic acid, eicosapentaenoic acid (EPA), docosahexaenoic acid (DHA), etc.

Examples of polyhydric alcohols include, for example, straight chain alcohols (such as lauryl alcohol, cetyl alcohol, stearyl alcohol, behenyl alcohol, myristyl alcohol, oleyl alcohol, and cetostearyl alcohol); and branched chain alcohols (such as monostearyl glycerin ether (batyl alcohol), 2-decyltetradecinol, lanolin alcohol, cholesterol, phytosterol, hexyldodecanol, isostearyl alcohol, and octyldodecanol), etc.

In addition, various types of surfactants other than the (E) component may be blended in the water based composition of the present disclosure as an emulsifier.

Examples of anionic surfactants include, for example, fatty acid soaps (sodium laurate, sodium palmitate, etc., for example); higher alkyl sulfate ester salts (sodium lauryl sulfate, potassium lauryl sulfate, etc.); alkyl ether sulfate ester salts (POE-triethanolamine lauryl sulfate, POE)-sodium lauryl sulfate, etc., for example); N-acylsarcosic acid (sodium lauroyl sarcosin, etc., for example); higher fatty acid amide sulfonate (N-myristyl-N-methyltaurine sodium, palm oil fatty acid methyltaurid sodium, lauryl methyl taurid sodium, etc., for example); phosphate ester salts (POE-oleyl ether phosphate sodium, POE-stearyl ether phosphate, etc.); sulfosuccinate (di-2-ethylhexyl sulfosuccinate sodium, mono lauroyl monoethanolamide poly oxyethylene, sodium lauryl polypropylene glycol sulfosuccinate, etc., for example); alkylbenzene sulfonate salt (sodium lina dodecylbenzene sulfonate, triethanolamine lina dodecylbenzene sulfonate, lina dodecylbenzene sulfate, etc., for example); higher fatty acid ester sulfate ester salt (hardened coconut oil fatty acid sodium glycerin sulfate, etc., for example); N-acyl glutamate (monosodium N-lauroyl glutamate, disodium N-stearoyl glutamate, monosodium N-myristoyl-L-glutamate, etc., for example); sulfated oil (scopolia oil, etc., for example); POE-alkyl ether carboxylic acid; POE-alkyl allyl ether carboxylic acid salt; α-olefin sulfonate; higher fatty acid ester sulfonate; secondary alcohol sulfate ester salt; higher fatty acid alkylol amide sulfate ester salt; sodium lauroyl monoethanolamide succinate; di triethanolamine N-palmitoyl aspartate; sodium caseinate, etc.

Examples of cationic surfactants include, for example, alkyl trimethylammonium salt (stearyl trimethylammonium chloride, lauryl trimethylammonium chloride, etc., for example); alkyl pyridinium salt (cetyl pyridinium chloride, etc., for example); distearyl dimethylammonium dialkyl dimethylammonium salt; poly (N,N′-dimethyl)-3,5-methylene piperidinium); alkyl quaternary ammonium salt; alkyl dimethyl benzyl ammonium salt; alkyl iso quinolinium salt; di alkyl moriphonium salt; POE-alkylamine; alkylamine salt; polyamine fatty acid derivative; amyl Examples thereof include alcohol fatty acid derivatives; benzalkonium chloride; benzethonium chloride, etc.

Examples of ampholytic surfactants include, for example, imidalozine series ampholytic surfactants (2-Undecyl-N,N,N-(hydroxyethylcarboxymethyl)-2-imidazoline sodium, 2-cocoyl-2-imidazolinium hydroxide-1-carboxyethyroxy disodium salt, etc.); betaine based surfactants (2-heptadecyl-N-carboxymethyl-N-hydroxyethyl imidazolinium betaine, lauryl dimethyl amino acetic acid betaine, alkyl betaine, amide betaine, sulfobetaine, etc.), etc.

Examples of lipophilic nonionic surfactants include, for example, sorbitan fatty acid esters (eg, sorbitan monooleate, sorbitan monoisostearate, sorbitan monolaurate, sorbitan monopalmitate, sorbitan monostearate, sorbitan sesquioleate, sorbitan trioleate, penta-2-ethylhexyl diglycerol) sorbitan, diglycerol sorbitan tetra-2-ethylhexylate, etc.); Glycerin polyglycerin fatty acids (mono cotton seed oil fatty acid glycerin, monoerkaate glycerin, sesquioleate glycerin, monostearate glycerin, a, a′-pyroglutamic acid oleate glycerin, glycerin malic acid monostearate, etc., for example); propylene glycol fatty acid esters (propylene glycol monostearate, etc.), for example; hardened castor oil derivatives; glycerin alkyl ether, etc.

Examples of lipophilic nonionic surfactants include, for example, sorbitan fatty acid esters (sorbitan monooleate, sorbitan monoisostearate, sorbitan monolaurate, sorbitan monopalmitate, sorbitan monostearate, sorbitan sesquioleate, sorbitan trioleate, diglycerol sorbitan penta-2-ethylhexylate, diglycerol sorbitan tetra-2-ethylhexylate, etc., for example); glycerin poly glycerin fatty acids (glycerin mono cotton seed oil fatty acid, mono erucic acid glycerin, glycerin sesquioleate, glycerin monostearate, glycerin a, a′-pyroglutamic acid oleate, glycerin malic acid monostearate, etc., for example); propylene glycol fatty acid esters (propylene glycol monostearate, etc., for example); hardened castor oil derivatives; glycerin alkyl ethers, etc.

Examples of hydrophilic nonionic surfactants include, for example, POE-sorbitan fatty acid esters (POE-sorbitan monooleate, POE-sorbitan monostearate, POE-sorbitan monooleate, POE-sorbitan tetraoleate, etc., for example); POE sorbit fatty acid esters (POE-sorbit monolaurate, POE-sorbit monooleate, POE-sorbit pentaoleate, POE-sorbit monostearate, etc., for example); POE-glycerin fatty acid esters (POE-monooleates such as POE-glycerin monostearate, POE-glycerin mono isostearate, POE-glycerin triisostearate, etc.); POE-fatty acid esters (POE-distearate, POE-monodiolate, ethylene glycol distearate, etc., for example); POE-alkyl ethers (POE-lauryl ether, POE-oleyl ether, POE-stearyl ether, POE-behenyl ether, POE-2-octyldodecyl ether, POE-cholestanol ether, etc., for example); pluronic types (for example, pluronic, etc.); POE⋅POP-alkyl ethers (POE⋅POP-cetyl ether, POE⋅POP-2-decyltetradecyl ether, POE⋅POP-monobutyl ether, POE⋅POP-hydrous lanolin, POE⋅POP-glycerin ether, etc., for example); tetra POE⋅tetra POP-ethylene diamine condensates (tetronic, etc., for example); POE-castor oil hardened castor oil derivatives (POE-castor oil, POE-hardened castor oil, POE-hardened castor oil mono isostearate, POE-hardened castor oil triisostearate, POE-hardened castor oil mono pyroglutamic acid mono isostearic acid diester, POE-hardened castor oil maleic acid, etc., for example); POE-beeswax⋅lanolin derivatives (POE-sorbit beeswax, etc., for example); alcanol amides (coconut oil fatty acid diethanol amide, laurate monoethanolamide, fatty acid isopropanol amide, etc., for example); POE-propylene glycol fatty acid esters; POE-alkylamines; POE-fatty acid amides; sucrose fatty acid esters; alkyl ethoxy dimethyl amine oxides; trioleyl phosphoric acid, etc.

A thickener other than the (D) component may be blended in the water based composition of the present disclosure. Specific examples of such thickeners include dextrin, sodium pectinate, sodium alginate, cellulose dialkyl dimethyl ammonium sulfate, aluminum magnesium silicate, bentonite, hectorite, AlMg silicate (bee gum), laponite, silicate anhydride, etc.

Examples of monosaccharides include, for example, trioses (D-glyceryl aldehyde, dihydroxyacetone, etc., for example); tetracarboses (eg, D-erythrose, D-erythrose, D-treose, etc.); pentoses (L-arabinose, D-xylose, L-lyxose, D-arabinose, D-ribose, D-ribrose, D-xylrose, L-xylrose, etc., for example); hexoses (D-glucose, D-talose, D-busicose, D-galactose, D-fructose, L-galactose, L-mannose, D-tagatose, etc., for example); heptoses (aldoheptose, heplose, etc., for example); octoses (octulose, etc., for example); deoxy sugars (2-deoxy-D-ribose, 6-deoxy-L-galactose, 6-deoxy-L-mannose, etc., for example); amino sugars (D-glucosamine, D-galactosamine, sialic acid, amino uronic acid, muramic acid, etc., for example); uronic acids (D-glucuronic acid, D-mannuronic acid, L-glulonic acid, D-galacturonic acid, L-isulonic acid, etc., for example).

Examples of oligosaccharides include, for example, sucrose, guntianose, umbelliferose, lactose, planteose, isolicnoses, α,α-trehalose, raffinose, lycnoses, umbilicin, stachyose velvascose, etc.

Examples of amino acids include, for example, neutral amino acids (threonine, cysteine, etc., for example); basic amino acids (hydroxylysine, etc., for example), etc. Examples of amino acid derivatives include, for example, sodium acyl sarcosine (sodium lauroyl sarcosine), acyl glutamate, sodium acyl β-alanine, glutathione, pyrrolidone carboxylic acid, etc.

Examples of organic amines include, for example, mono ethanolamine, diethanolamine, triethanolamine, morpholine, tri iso propanolamine, 2-amino-2-methyl-1,3-propanediol, 2-amino-2-methyl-1-propanol. etc.

Examples of alkylene oxide derivatives include, for example, POE (9) POP (2) dimethyl ether, POE (14) POP (7) dimethyl ether, POE (10) POP (10) dimethyl ether, POE (6) POP (14) dimethyl ether, POE (15) POP (5) dimethyl ether, POE (25) POP (25) dimethyl ether, POE (7) POP (12) dimethyl ether, POE (22) POP (40) dimethyl ether, POE (35) POP (40) dimethyl ether, POE (50) POP (40) dimethyl ether, POE (55) POP (30) dimethyl ether, POE (30) POP (34) dimethyl ether, POE (25) POP (30) dimethyl ether, POE (27) POP (14) dimethyl ether, POE (55) POP (28) dimethyl ether, POE (36) POP (41) dimethyl ether, POE (7) POP (12) dimethyl ether, POE (17) POP (4) dimethyl ether, etc.

Examples of metal ion sequestering agents include, for example, 1-hydroxyethane-1,1-diphosphonic acid, 1-hydroxyethane-1,1-diphosphonic acid tetrasodium salt, disodium edetate, trisodium edetate, tetrasodium edetate, sodium citrate, sodium polyphosphate, sodium metaphosphate, gluconic acid, phosphoric acid, citrate, ascorbic acid, succinic acid, edetic acid, trisodium ethylene diamine hydroxyethyl triacetate, etc.

Examples of antioxidizing aiding agents include phosphoric acid, citric acid, ascorbic acid, maleic acid, malonic acid, succinic acid, fumaric acid, kephalin, hexametaphosphate, phytic acid, ethylenediamine tetraacetic acid, etc.

Other components which may be blended include, for example, preservatives (ethyl paraben, butylparaben, etc.); whitening agents (placenta extract, saxifrage extract, arbutin, etc., for example); blood circulation promoters (nicotinic acid, benzyl nicotinate, tocopherol nicotinate, β-butoxy ester nicotinate, minoxidyl or its relatives, γ-orizanol, alkoxy carbonyl pyridine N-oxide, carpronium chloride, acetylcholine or derivatives thereof, etc.); various extracts (ginger, phellodendron, coptis, lithospermum, birch, loquat, carrot, aloe, mallow, iris, grape, loofah, lily, saffron, cnidium, zingiber, hypericum, ononis, garlic, capsicum, citrus peel, Japanese angelica, peony, seaweed, etc., for example); activator agents (pantenyl ethyl ether, nicotinic acid amide, biotin, pantothenic acid, royal jelly, cholesterol derivatives, etc., for example); antilipolytic agents (pyridoxins, thiantoll, etc., for example), etc.

Although the viscosity of the water based composition of the present disclosure will differ depending on the form of the composition, it is preferable for the viscosity to be within a range from 10,000 to 100,000 mPa·s. By the viscosity of the composition being greater than or equal to 10,000 mPa·s, preparations can be maintained in a stable manner, and by the viscosity of the composition being less than or equal to 100,000 mPa·s, the composition can be uniformly coated on skin, etc.

The form of the water based composition of the present disclosure is not particularly limited, and the composition may be applied to a wide range of forms which are conventionally employed as cosmetics. Such forms include base cosmetics such as lotions, milky lotions, creams, facial cleansers, gels, essences, facial masks, etc.; makeup cosmetics such as lipstick, eye shadow, eyeliner, mascara, foundation, sunscreen, etc.; oral cosmetics; aromatic cosmetics; hair cosmetics; body cosmetics; etc.

### Examples

The present disclosure will be described in greater detail by the Examples below. However, the present disclosure is not limited to these Examples. In addition, the contents of each component in the Examples below are percent by mass, unless noted otherwise.

Water based compositions were produced by standard methods according to the preparations shown in Tables 1 and 2 below, and were evaluated by the following standards.

### (Viscosity)

The viscosities of the water based compositions were measured at 30° C. with a BL type viscometer (Model VS-A by Shibaura Systems). The measurement conditions were such that the compositions were stirred with Rotor Number 3 in cases that the viscosity was less than 10,000 mPa·s, and stirred with Rotor Number 4 in cases that the viscosity was greater than or equal to 10,000 mPa·s for one minute at 12 rpm.

### (Dispersion Properties)

The water based compositions which were prepared were placed in 50 ml sample tubes (diameter: 3 cm), rotated for four hours at a speed of 45 rpm at a temperature of 25° C. to conduct rolling tests. The degree of powder agglomeration was visually confirmed, and evaluated by the standards below.

A: No agglomeration or precipitation
B: Some agglomeration or precipitation
C: Agglomeration and precipitation present

### (Lasting Cosmetic Effect)

A panel of ten experts utilized the prepared water based compositions and evaluated the lasting cosmetic effects thereof. The evaluation standards were as follows.

### <Evaluation Points>

5 points: Extremely favorable
4 points: Favorable
3 points: Fair
2 points: Poor
1 point: Extremely poor

### <Evaluation Standards>

A: Average of points is greater than or equal to 4.5
B: Average of points is greater than or equal to 3.5 and less than 4.5
C: Average of points is greater than or equal to 2.5 and less than 3.5
D: Average of points is less than 2.5

The preparations and evaluation results are shown in Tables 1 and 2. The details of the main components (commercially available products) are as follows.
Methyl Methacrylate Cross Polymer (porous): Techpolymer MBP-8HP (by Sekisui Kasei Industries Co.)
Methyl Methacrylate Cross Polymer (non porous): Ganz Pearl GMX-0810 (by Aica Kogyo Co.)
Hydrophobic Silica: Porous Silica Beads SA-SB-705 (by Miyoshi Kasei Co.)
(Vinyl Dimethicone/Methicone Silsesquioxane) Cross Polymer: Silicone Powder KSP102 (by Shin Etsu Chemical Industries Co.)
Cornstarch: Starch C
Silica: Sunsphere L-51S (by AGC Si Tech Co.)
Dimethicone/Dimethicone Cross Polymer: Elastomer Blend DC9041 (dimethicone cross polymer containing product (pure component: 16%)) (by Toray Dow Corning Co.)
(Acryloyl Dimethyl Taurine Ammonium/VP) Copolymer: Aristoflex AVC (by Clariant Japan Co.)
(Acrylates/Alkyl Acrylate (C10-C30)) Copolymer: PEMULEN TR-2 (by Lubrizol Advanced Materials, Inc.)
Polysorbate 20: Nikkol TL-10 (by Nikko Chemicals Co.)

TABLE 1

| Type of | | | | | | Comparative | Comparative | Comparative | Comparative |
|---|---|---|---|---|---|---|---|---|---|
| Component | All Components | Example 1 | Example 2 | Example 3 | Example 4 | Example 1 | Example 2 | Example 3 | Example 4 |
| (C) Aqueous | Ion Exchanged Water | to 100% | to 100% | to 100% | to 100% | to 100% | to 100% | to 100% | to 100% |
| Medium | | | | | | | | | |
| Powder | Methyl Methacrylate | 10 | 10 | 10 | | | | | |
| | Cross Polymer | | | | | | | | |
| | (Porous) | | | | | | | | |
| | Methyl Methacrylate | | | | | | | | 10 |
| | Cross Polymer | | | | | | | | |
| | (Non Porous) | | | | | | | | |
| | Hydrophobic Silica | | | | 10 | | | | |
| | (Vinyl Dimethicone/ | | | | | 10 | | | |
| | Methicone | | | | | | | | |
| | Silsequioxane) | | | | | | | | |
| | Cross Polymer | | | | | | | | |
| | Cornstarch | | | | | | 10 | | |
| | Silica | | | | | | | 10 | |
| (A) Crosslinked | Dimethicone | 17 | 13 | 18.5 | 17 | 17 | 17 | 17 | 17 |
| Siloxane | (Dimethicone/ | | 3 | | | | | | |
| Elastomer | (PEG-10/15)) | | | | | | | | |
| | Cross Polymer | | | | | | | | |
| | (Dimethicone/Vinyl | | | 1.5 | | | | | |
| | Dimethicone) | | | | | | | | |
| | Cross Polymer | | | | | | | | |
| | Dimethicone | 3 | 3 | 3 | 3 | 3 | 3 | 3 | 3 |
| | Cross Polymer | | | | | | | | |
| (D) Water | (Acryloyl Dimethyl | 0.3 | 0.3 | 0.3 | 0.3 | 0.3 | 0.3 | 0.3 | 0.3 |
| Soluble | Taurine Ammonium/VP) | | | | | | | | |
| Thickener | Copolymer | | | | | | | | |
| | (Acrylates/Alkyl | 0.03 | 0.03 | 0.03 | 0.03 | 0.03 | 0.03 | 0.03 | 0.03 |
| | Acrylate (C10-C30)) | | | | | | | | |
| | Cross Polymer | | | | | | | | |
| Surfactant | Polysorbate 20 | 1.8 | 1.8 | 1.8 | 1.8 | 1.8 | 1.8 | 1.8 | 1.8 |

| | | | | | | | | |
|---|---|---|---|---|---|---|---|---|
| Viscosity | 50000 ↑ | 45990 | 45300 | 33100 | 32270 | 11010 | 15520 | 9960 |
| Lasting Cosmetic Effect | A | A | A | B | C | C | C | C |
| Dispersion Properties | A | A | A | A | B | B | A | A |

TABLE 2

| Type of | | | | | | Comparative | Comparative | Comparative |
|---|---|---|---|---|---|---|---|---|
| Component | All Components | Example 1 | Example 5 | Example 6 | Example 7 | Example 5 | Example 6 | Example 7 |
| (C) Aqueous | Ion Exchanged Water | to 100% | to 100% | to 100% | to 100% | to 100% | to 100% | to 100% |
| Medium | | | | | | | | |
| (B) Porous | Methyl Methacrylate | 10 | 7.5 | 5 | 2.5 | | 2.5 | |
| Powder | Cross Polymer | | | | | | | |
| | (Porous) | | | | | | | |
| (A) Crosslinked | Dimethicone | 17 | 17 | 17 | 17 | 17 | | |
| Siloxane | Dimethicone Cross | 3 | 3 | 3 | 3 | 3 | | |
| Elastomer | Polymer | | | | | | | |
| (D) Water | (Acryloyl Dimethyl | 0.3 | 0.3 | 0.3 | 0.3 | 0.3 | 0.3 | 0.3 |
| Soluble | Taurine Ammonium/VP) | | | | | | | |
| Thickener | (Acrylates/Alkyl | 0.03 | 0.03 | 0.03 | 0.03 | 0.03 | 0.03 | 0.03 |
| | Acrylate (C10-C30)) | | | | | | | |
| | Cross Polymer | | | | | | | |
| Surfactant | Polysorbate 20 | 1.8 | 1.8 | 1.8 | 1.8 | 1.8 | 1.8 | 1.8 |

| | | | | | | | |
|---|---|---|---|---|---|---|---|
| Viscosity | 50000 ↑ | 22790 | 14050 | 10360 | 5910 | 3940 | 3310 |
| Lasting Cosmetic Effect | A | A | A | A | C | A | C |
| Dispersion Properties | A | A | A | A | A | A | A |
| Ratio of (B) Porous Powder with | 0.14 | 0.10 | 0.07 | 0.03 | — | — | — |
| respect to (C) Aqueous Medium | | | | | | | |

As is shown in Table 1, the water based compositions of Examples 1 through 4 had superior, higher viscosity, longer lasting cosmetic effects, and favorable dispersion properties compared to Comparative Examples 1 through 4 that utilized different types of powders.

Examples 5 through 7 of Table 2 are those in which the amount of the (B) component was changed from that of Example 1. The Example 7, which had the least amount of the (B) component exhibited a superior, higher viscosity than Comparative Example 5 which does not contain the (B) component and Comparative Example 6 which does not contain the (A) component. In addition, Examples 1 and 5, in which the ratio of the crosslinked siloxane elastomer with respect to the aqueous medium is greater than or equal to 0.1 had significantly higher viscosities.

Next, oil in water type compositions were produced by standard methods according to the preparations shown in Table 3 below, and were evaluated by the following standards.

### (Stability)

The states of the prepared oil in water type compositions, which were stored in 50 ml glass bottles at room temperature for four months, were visually confirmed.

A: No visible separation
B: A slight transparent layer is visible
C. A transparent layer of greater than or equal to 1/10 of the total amount is visible

The preparations and evaluation results are shown in Table 3. Note that the details of the main components (commercially available products) are as follows.
Isostearate PEG-60 Glyceryl: Emalex GWIS-160N (by Nippon Emulsion Co.)
Isostearate PEG-20 Glyceryl: Emalex GWIS-120 (by Nippon Emulsion Co.)
PPG-8 Cetus-20: Nikkol PBC-44 (by Nikko Chemicals Co.)
PPG-4 Cetus-10: Nikkol PBC-33 (by Nikko Chemicals Co.)
PPG48 Cetus-20: Nikkol PBC-34 (by Nikko Chemicals Co.)

TABLE 3

| | | | Example | Example | Example | Example | Example | Example |
|---|---|---|---|---|---|---|---|---|
| TYPE OF COMPONENT | ALL COMPONENTS | HLB | 8 | 9 | 10 | 11 | 12 | 13 |
| (C) Aqueous Medium | Water | | to 100% | to 100% | to 100% | to 100% | to 100% | to 100% |
| | Glycerin | | 2 | 2 | 2 | 2 | 2 | 2 |
| | DPG | | 8 | 8 | 8 | 8 | 8 | 8 |
| (D) Water Soluble | (Acryloyl Dimethyl Taurine | | 0.5 | 0.5 | 0.5 | 0.5 | 0.5 | 0.5 |
| Thickener | Ammonium/VP) Copolymer | | | | | | | |
| Surfactant | PEG-60 Hydrogenated Castor Oil | 14 | | | | | | |
| | PEG-100 Hydrogenated Castor Oil | 16.5 | | | | | | |
| | PEG-40 Hydrogenated Castor Oil | 12.5 | | | | | | |
| | PEG-60 Glyceryl Isostearate | 18.3 | | | | | | |
| | PEG-20 Glyceryl Isostearate | 13 | 1 | 1 | 1 | | | |
| | PPG-8 Cetus 20 | 12.5 | | | | 1 | | |
| | PPG-4 Cetus 10 | 10.5 | | | | | 1 | |
| | PPG-4 Cetus 20 | 16.5 | | | | | | 1 |
| | Behenes-10 | 10 | | | | | | |
| | Behenes-20 | 16.5 | | | | | | |
| (F) UV Absorber | Ethyl Hexyl Methoxycinnamate | | 7.5 | 7.5 | 7.5 | 7.5 | 7.5 | 7.5 |
| | t-Butyl Methoxy Dibenzoyl | | 2.5 | 2.5 | 2.5 | 2.5 | 2.5 | 2.5 |
| | Methane | | | | | | | |
| | Octocrylene | | 3 | 3 | 3 | 3 | 3 | 3 |
| | Homosalate | | 5 | 5 | 5 | 5 | 5 | 5 |
| | PPG-17 | | 1 | 1 | 1 | 1 | 1 | 1 |
| | Isostearate | | 0.2 | 0.2 | 0.2 | 0.2 | 0.2 | 0.2 |
| (B) Porous Powder | Methyl Methacrylate Cross | | 5 | 5 | 5 | 5 | 5 | 5 |
| | Polymer | | | | | | | |
| Surfactant | Polysorbate 20 | | 1.8 | 1.8 | 1.8 | 1.8 | 1.8 | 1.8 |
| (A) Crosslinked | Dimethicone | | 25 | 23 | 28 | 25 | 25 | 25 |
| Siloxane | (Dimethicone/(PEG-10/15)) | | | 7 | | | | |
| Elastomer | Cross Polymer | | | | | | | |
| | (Dimethicone/Vinyl | | | | 2 | | | |
| | Dimethicone) Cross Polymer | | | | | | | |
| | Dimethicone Cross Polymer | | 4.5 | | | 4.5 | 4.5 | 4.5 |

| | | | | | | |
|---|---|---|---|---|---|---|
| Stability | A | A | A | A | A | A |

| | | | Test | Test | Test | Test | Test | Test |
|---|---|---|---|---|---|---|---|---|
| TYPE OF COMPONENT | ALL COMPONENTS | HLB | Sample 1 | Sample 2 | Sample 3 | Sample 4 | Sample 5 | Sample 6 |
| (C) Aqueous Medium | Water | | to 100% | to 100% | to 100% | to 100% | to 100% | to 100% |
| | Glycerin | | 2 | 2 | 2 | 2 | 2 | 2 |
| | DPG | | 8 | 8 | 8 | 8 | 8 | 8 |
| (D) Water Soluble | (Acryloyl Dimethyl Taurine | | 0.5 | 0.5 | 0.5 | 0.5 | 0.5 | 0.5 |
| Thickener | Ammonium/VP) Copolymer | | | | | | | |
| Surfactant | PEG-60 Hydrogenated Castor Oil | 14 | 1 | | | | | |
| | PEG-100 Hydrogenated Castor Oil | 16.5 | | 1 | | | | |
| | PEG-40 Hydrogenated Castor Oil | 12.5 | | | 1 | | | |
| | PEG-60 Glyceryl Isostearate | 18.3 | | | | 1 | | |
| | PEG-20 Glyceryl Isostearate | 13 | | | | | | |
| | PPG-8 Cetus 20 | 12.5 | | | | | | |
| | PPG-4 Cetus 10 | 10.5 | | | | | | |
| | PPG-4 Cetus 20 | 16.5 | | | | | | |
| | Behenes-10 | 10 | | | | | 1 | |
| | Behenes-20 | 16.5 | | | | | | 1 |
| (F) UV Absorber | Ethyl Hexyl Methoxycinnamate | | 7.5 | 7.5 | 7.5 | 7.5 | 7.5 | 7.5 |
| | t-Butyl Methoxy Dibenzoyl | | 2.5 | 2.5 | 2.5 | 2.5 | 2.5 | 2.5 |
| | Methane | | | | | | | |
| | Octocrylene | | 3 | 3 | 3 | 3 | 3 | 3 |
| | Homosalate | | 5 | 5 | 5 | 5 | 5 | 5 |
| | PPG-17 | | 1 | 1 | 1 | 1 | 1 | 1 |
| | Isostearate | | 0.2 | 0.2 | 0.2 | 0.2 | 0.2 | 0.2 |
| (B) Porous Powder | Methyl Methacrylate Cross | | 5 | 5 | 5 | 5 | 5 | 5 |
| | Polymer | | | | | | | |
| Surfactant | Polysorbate 20 | | 1.8 | 1.8 | 1.8 | 1.8 | 1.8 | 1.8 |
| (A) Crosslinked | Dimethicone | | 25 | 25 | 25 | 25 | 25 | 25 |
| Siloxane | (Dimethicone/(PEG-10/15)) | | | | | | | |
| Elastomer | Cross Polymer | | | | | | | |
| | (Dimethicone/Vinyl | | | | | | | |
| | Dimethicone) Cross Polymer | | | | | | | |
| | Dimethicone Cross Polymer | | 4.5 | 4.5 | 4.5 | 4.5 | 4.5 | 4.5 |

| | | | | | | |
|---|---|---|---|---|---|---|
| Stability | C | C | C | C | C | C |

As is shown in Table 3, in the case that a UV ray absorber is contained as the (F) polar oil component, stability was favorable in Examples 8 through 13, in which the surfactant is a poly oxyethylene-poly oxy alkylene alkyl ether block polymer or a poly oxyethylene fatty acid glyceryl having HLB values within a range from 9 to 17. In contrast, a satisfactory level of stability was not obtained in cases that surfactants having HLB values outside the above range were contained.

An example of a preparation of the water based composition is shown below.

### Preparation Example 1: Oil in Water Type Foundation

| Ingredient | Percent by Mass |
|---|---|
| Water | Remainder |
| Glycerin | 2 |
| Di Propylene Glycol | 8 |
| (Acryloyl Dimethyl Taurine Ammonium/VP) | 0.5 |
| Copolymer | |
| (Acryloyl Dimethyl Taurine Ammonium/Behenes | |
| Methacrylate-25) Cross Polymer 0.5 | |
| Methyl Methacrylate Cross Polymer | 5 |
| Polysorbate 20 | 2 |
| PEG-10 Dimethicone | 1 |
| Dimethicone Cross Polymer | 5 |
| PPG-8 Cetus-20 | 0.5 |
| PEG-60 Hydrogenated Castor Oil | 0.1 |
| PEG-20 Glyceryl Isostearate | 1 |
| PPG-17 | 1 |
| Isostearate | 0.2 |
| Ethyl hexyl Methoxycinnamate | 7.5 |
| Ethyl Hexyl Salicylate | 5 |
| t-Butyl Methoxy Dibenzoyl Methane | 2.5 |
| Octocrylene | 3 |
| Dimethicone | 28 |
| Iron Oxide | 0.01 |
| Ba Sulfate | 1 |
| Glycyl Glycine | 2 |
| Na Metaphosphate | 0.1 |
| Phenoxy Ethanol | 0.5 |
| Methyl Paraben | 0.15 |
| Ethyl Paraben | 0.1 |

1. A water based composition, comprising:
an (A) crosslinked siloxane elastomer;
a (B) lipophilic porous powder; and
a (C) aqueous medium.

2. The water based composition as defined in claim 1, wherein crosslinked polymethyl methacrylate is the main component of the (B) lipophilic porous powder.

3. The water based composition as defined in either one of claim 1, further comprising:
a (D) water soluble thickener.

4. The water based composition as defined in claim 1, further comprising:
a (E) surfactant; wherein
the surfactant is poly oxyethylene-poly oxy alkylene alkyl ether block polymer or poly oxyethylene fatty acid glyceryl having an HLB value within a range from 9 to 17.

5. The water based composition as defined in claim 4, further comprising:
a (F) polar oil component.

6. The water based composition as defined in claim 5, wherein:
the (F) polar oil component is a UV absorber.

7. The water based composition as defined in claim 1, wherein:
the ratio of the amount of the (B) lipophilic porous powder with respect to the amount of the (C) aqueous medium is greater than or equal to 0.01.

8. The water based composition as defined in claim 7, wherein:
the ratio of the amount of the (B) lipophilic porous powder with respect to the amount of the (C) aqueous medium is greater than or equal to 0.1.

9. The water based composition as defined in claim 1, wherein:
the composition is of the oil in water type.
